(19) United States
(12) Patent Application Publication
(10) Pub. No.: US 2002/0116064 A1
(43) Pub. Date: Aug. 22, 2002

(54) Apparatus for fusing adjacent bone structures

(76) Inventor: Lance Middleton, Trumbull, CT (US)

Correspondence Address: United States Surgical,, a Division of Tyco Healthcare Group, LP, 150 Glover Avenue, Norwalk, CT, 06856, US

(21) Appl. No.: 09/790,043

(22) Filed: Feb. 21, 2001

(51) Int. Cl. A61B 17/70

(52) U.S. Cl. 623/17.16

(57) ABSTRACT

An apparatus for facilitating fusion of adjacent bony structures includes an implant body dimensioned for positioning between adjacent bone structures to maintain the bone structures in desired spaced relation during interbody fusion. The implant body defines a longitudinal axis and has an outer wall extending along the longitudinal axis. The outer wall includes a plurality of annular serrated portions spaced along the longitudinal axis. The annular serrated portions are dimensioned and configured to engage the adjacent bone structures to facilitate retention of the implant member therewithin. The implant member further include at least one concave surface at least partially extending along the longitudinal axis, whereby the transverse cross-sectional dimension along a first transverse axis inclusive of the concave surface is less than the transverse cross-sectional dimension along a second transverse axis. The concave surface extends substantially along the length of the implant member. Preferably, the implant body includes a pair of diametrically opposed concave wall surfaces.

### BACKGROUND

1. Technical Field

The present disclosure generally relates to a surgical apparatus for fusing adjacent bone structures, and, more particularly, to an apparatus and associated method for fusing adjacent vertebrae.

2. Background of the Related Art

The fusion of adjacent bone structures is commonly performed to provide for long-term replacement to compensate for degenerative or deteriorated disorders in bone. For example, an intervertebral disc, which is a ligamentous cushion disposed between adjacent vertebrae, may undergo deterioration as a result of injury, disease, tumor or other disorders. The disk shrinks or flattens leading to mechanical instability and painful disc translocations.

Conventional procedure for disc surgery include partial or total excision of the injured disc portion, e.g., discectomy, and replacement of the excised disc with biologically acceptable plugs or bone wedges. The plugs are driven between adjacent vertebrae to maintain normal intervertebral spacing and to achieve, over a period of time, bony fusion with the plug and opposed vertebrae. More recently, emphasis has been placed on fusing bone structures (i.e., adjoining vertebrae) with metallic or ceramic prosthetic cage implants. One fusion cage implant is disclosed in commonly assigned U.S. Pat. No. 5,026,373 to Ray et al., the contents of which are incorporated herein by reference. The Ray '373 fusion cage includes a cylindrical cage body having a thread formed as part of its external surface and apertures extending through its wall which communicate with an internal cavity of the cage body. The fusion cage is inserted within a tapped bore or channel formed in the intervertebral space thereby stabilizing the vertebrae and maintaining a pre-defined intervertebral space. Preferably, a pair of fusion cages are implanted within the intervertebral space. The adjacent vertebral bone structures communicate through the apertures and with bone growth inducing substances which are within the internal cavity to unite and eventually form a solid fusion of the adjacent vertebrae. FIGS. 1-2 illustrate the insertion of a pair of the Ray '373 fusion cages positioned within an intervertebral space.

### SUMMARY OF THE INVENTION

Accordingly, the present invention is directed to further improvements in spinal fusion procedures. In accordance with a preferred embodiment, an apparatus for facilitating fusion of adjacent bone structures includes an implant body dimensioned for positioning between adjacent bone structures to maintain the bone structures in desired spaced relation during interbody fusion. The implant body defines a longitudinal axis and has an outer wall extending along the longitudinal axis. The outer wall includes a plurality of annular serrated portions spaced along the longitudinal axis. The annular serrated portions are dimensioned and configured to engage the adjacent bone structures to facilitate retention of the implant member therewithin. The implant member includes at least one concave wall surface at least partially extending along the longitudinal axis wherein the transverse cross-sectional dimension along a first transverse axis inclusive of the concave wall surface is less than the transverse cross-sectional dimension along a second transverse axis. The concave wall surface advantageously reduces the transverse cross-sectional dimension of the implant member thereby facilitating placement of the implant member in restricted intervertebral areas. In addition, the concave wall surface facilitates placement of a pair of implants in side-by-side relation. Preferably, the implant body includes a pair of diametrically opposed concave wall surfaces thereby further reducing the cross-sectional dimension of the implant. A method for facilitating fusion of adjacent vertebrae is also disclosed.

### BRIEF DESCRIPTION OF THE DRAWINGS

Preferred embodiment(s) of the present disclosure are described herein with reference to the drawings wherein:

FIG. 1 is a view illustrating a portion of the vertebral column of a patient;

FIG. 2 is a view taken along line 2-2 of FIG. 1 illustrating a pair of prior art fusion implants positioned within the intervertebral space for fusion of adjacent vertebrae;

FIG. 3 is a perspective view of the fusion implant apparatus in accordance with the principles of the present disclosure;

FIG. 4 is a side plan view of the implant apparatus;

FIG. 5 is a view illustrating details of the annular serrations of the implant apparatus;

FIG. 6 is an axial view of the implant apparatus;

FIG. 7 is an axial cross-sectional view of the implant apparatus taken along the lines 7-7 of FIG. 4;

FIG. 8 is a side cross-sectional view of the implant apparatus taken along the lines 8-8 of FIG. 6; and

FIGS. 9-11 are views illustrating preferred sequences of insertion of the implant apparatus within adjacent vertebrae.

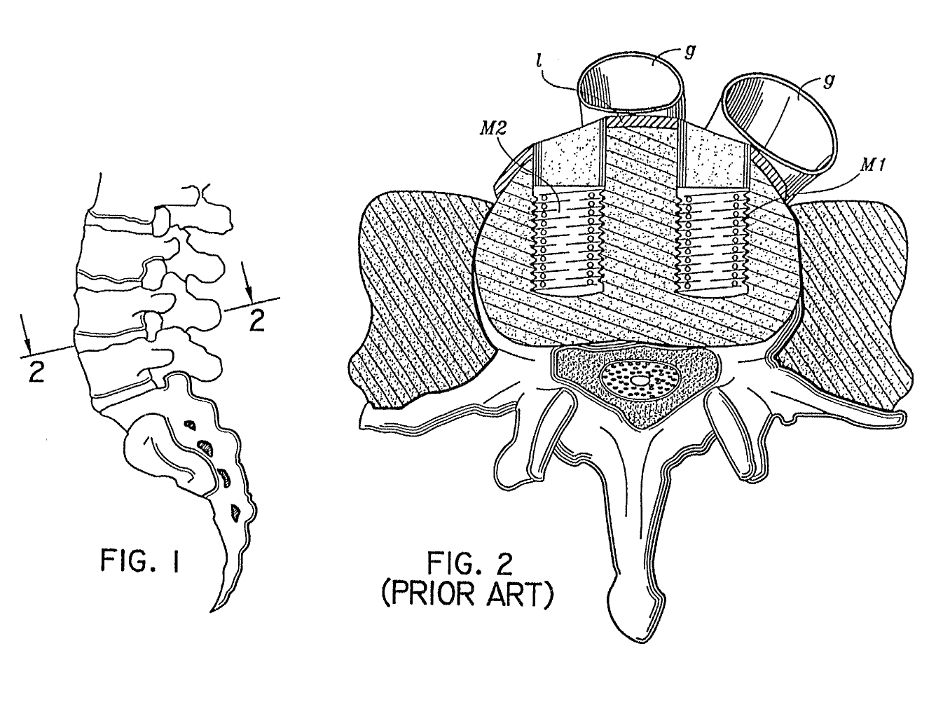

### DETAILED DESCRIPTION OF PREFERRED EMBODIMENTS

The preferred embodiment of the apparatus and method disclosed herein are discussed in terms of orthopedic spinal fusion procedures and instrumentation. It is envisioned, however, that the disclosure is applicable to a wide variety of procedures including, but, not limited to ligament repair, joint repair or replacement, non-union fractures, facial reconstruction and spinal stabilization. In addition, it is believed that the present method and instrumentation finds application in both open and minimally invasive procedures including endoscopic and arthroscopic procedures wherein access to the surgical site is achieved through a cannula or small incision.

The following discussion includes a description of the fusion implant utilized in performing a spinal fusion followed by a description of the preferred method for spinal fusion in accordance with the present disclosure.

In the discussion which follows, the term “proximal”, as is traditional, will refer to the portion of the structure which is closer to the operator while the term “distal” will refer to the portion which is further from the operator.

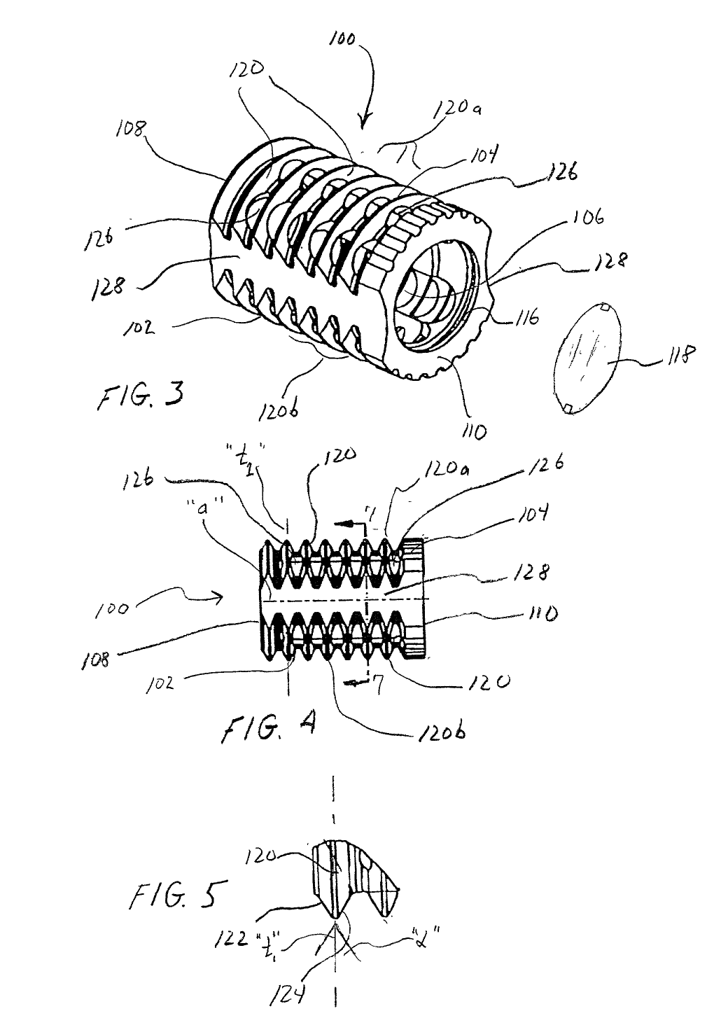

Referring now to the drawings in which like reference numerals identify similar or identical elements throughout the several views, FIG. 3 illustrates, in perspective, the fusion implant apparatus of the present disclosure. Fusion implant 100 is intended to be inserted within a preformed bore in adjacent bone structures, e.g., adjacent vertebrae, in a bore spanning the intervertebral space without necessitating rotation of the implant for placement within the vertebrae, i.e., the fusion implant 100 is not threaded, but, however, incorporates novel structure which positively secures the implant 100 within the adjacent vertebrae.

Fusion implant 100 includes elongated implant body 102 which is preferably fabricated from a suitable biocompatible rigid material such as titanium and/or alloys of titanium, stainless steel, ceramic materials or rigid polymeric materials. Implant body 102 is preferably sufficient in strength to at least partially replace the supporting function of an intervertebral disc, i.e., to maintain adjacent vertebrae in desired spaced relation, during healing and fusion.

With reference to FIGS. 3-8, implant body 102 includes exterior or outer wall 104 concentrically arranged about longitudinal axis “a” of the implant body 102 and inner cavity 106 defined within the exterior wall 104. Implant body 102 is preferably substantially cylindrical in configuration defining a constant diameter along its length. Inner cavity 106 is intended to accommodate bone growth inducing substances such as bone chips taken from allograft or autograft, etc . . . which facilitate the fusion process as is conventional in the art. Implant body 102 is preferably provided in various lengths ranging from about 18 mm-24 mm and in corresponding various diameters ranging from about 14 mm-18 mm. Other dimensions are also contemplated and may vary depending on the intended use of the implant in the cervical, thoracic or lumbar regions of the spine.

Implant body 102 defines entry and trailing end faces 108, 110. End faces 108,110 are preferably open, i.e, having respective apertures 112, 114 therein in communication with the inner cavity 106. As best depicted in FIG. 8, implant body 102 has internal annular recesses 116 adjacent each end face 108,110. Annular recesses 116 are intended to receive plastic end caps 118 (FIG.3; only one end cap 118 is shown) which are received within the recesses in snap-fit relation therewith to enclose inner cavity 106 thereby retaining the bone growth inducing substances therein.

Outer wall 104 has a plurality of annular serrated portions 120 equidistally spaced along the longitudinal axis “a” of implant body 102. Annular serrated portions 120 extend generally transverse to the longitudinal axis “a” of implant body 102. Annular serrated portions 120 preferably include a first group 120a of serrated portions 120 and a second group 120b of serrated portions 120 in diametrical opposed relation to the first group 120a. Each serrated portion 120 of the two groups 120a, 120b preferably subtends an angle less than about 180° relative to the longitudinal axis “a”.

Each serrated portion 120 further defines a leading surface 122 and a trailing surface 124 with respect to the leading and trailing end faces 108,110 of the implant body 102. Leading and trailing surfaces 122,124 of serrated portions 120 are obliquely arranged with respect to the longitudinal axis defining an angle ranging from about 20° to 40°, preferably, 30°, relative to the axis “t1” transverse to the longitudinal axis. Serrated portions 120 are advantageously dimensioned to lockingly engage the adjacent vertebrae upon insertion within the intervertebral space to secure implant body 102 therewithin. Moreover, the oblique arrangement of the leading and trailing surfaces 122, 124 become embedded within the vertebrae bone and, by virtue of their inclined arrangement, resist movement in either the leading or trailing directions, thereby positively retaining the implant body within the adjacent vertebrae.

A plurality of apertures 126 extend through outer wall 104 of implant body 102. Apertures 126 are preferably formed by broaching grooves in the internal surface of the inner cavity 106. The effect of such broaching is to remove material from the valleys defined between adjacent serrations 120, thus defining the apertures 126. The advantages of such an arrangement are disclosed in U.S. Pat. No. 4,961,740, the contents of which are incorporated herein by reference, and include immediate bone to bone contact between the vertebral bodies or bone structures and the bone inducing substances packed within the inner cavity 106 of the implant body 102. Apertures 126 are preferably substantially the same in dimension although it is envisioned that the dimensions of the apertures may vary to provide for more or less bone to bone contact as desired.

As best depicted in FIGS. 3, 4 and 7, apertures 126 are clustered about transverse axis “t1”, both at the upper and lower end of the axis. Consequently, apertures 126 come into contact with the upper and lower vertebral bone structures to encourage bone growth through implant body 102 from the vertebral bone structures when appropriately positioned within the vertebrae. The lateral sections of implant body 102 formed along transverse axis “t2” do not have apertures in order to prevent growth of disk material which might interfere with the bone fusion process.

Outer wall 104 further includes diametrically opposed arcuate surfaces 128 defined in the outer wall and extending along the length of implant body 102. Each arcuate surface 128 is preferably concave in configuration and may be formed by grinding, blasting applications, etc.

Preferably, concave surfaces 128 extend radially inwardly within each serration 120 thereby defining removed portions of the serrations 120 as shown. Concave surfaces 128 interconnect the opposed serrated portions 120a, 120b.

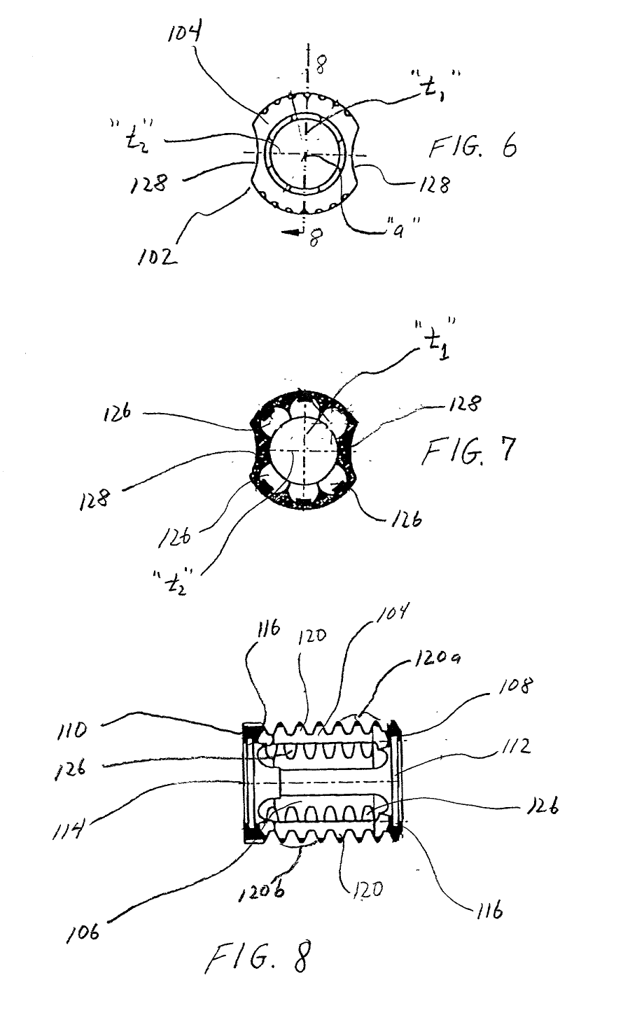

The concave surface arrangement provides two specific advantages. First, such arrangement increases the pull out or expulsion force necessary to remove the implant from the adjacent vertebrae. Secondly, the concave surface arrangement permits a pair of implants to be positioned in side by side relation within the adjacent vertebrae. Moreover, the concave surface arrangement provides a reduced cross-sectional dimension along second transverse axis “t2” relative to the cross-sectional dimension along first transverse axis “t1” thereby facilitating placement of the implant body 102 within restricted vertebral locations. (FIG. 6) Preferably, the transverse cross-sectional dimension along transverse axis “t2” is 20-40% less than the transverse cross-sectional dimension along transverse axis “t1”.

Insertion of Fusion Implant

The insertion of the fusion implant 100 into an intervertebral space defined between adjacent lumbar vertebrae will now be described. The subsequent description will be particularly discussed in conjunction with an open posterior approach for spinal fusion implant insertion. However, it is to be appreciated that other approaches, e.g., anterior, lateral, posterior lateral, anterior lateral etc . . . could be utilized. Laparoscopic approaches are also envisioned.

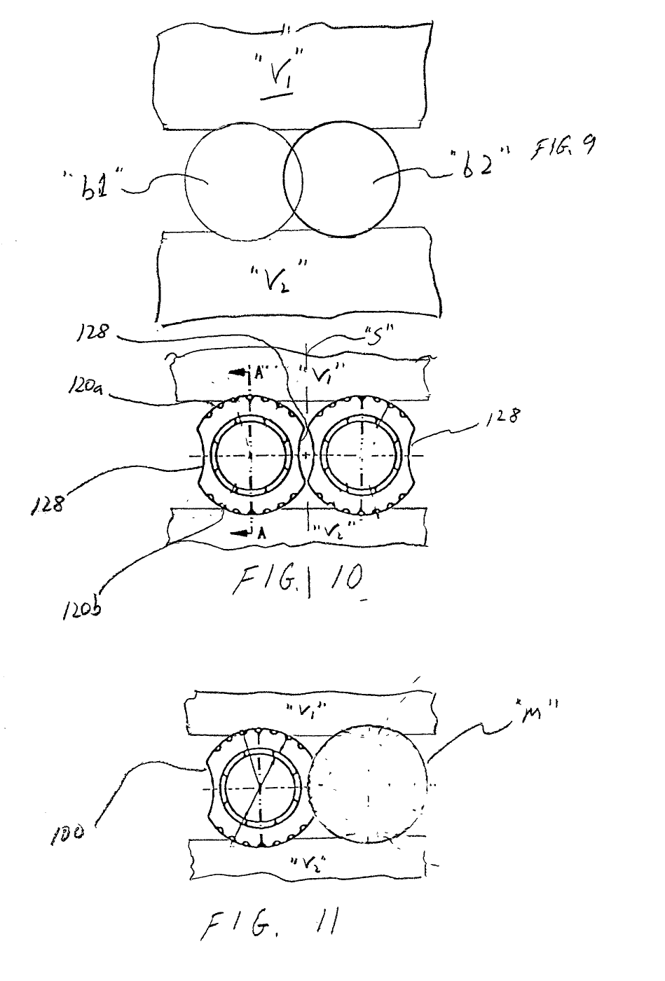

Initially, a first lateral side of the intervertebral space “i” is accessed utilizing appropriate retractors to expose the posterior vertebral surface. A drilling instrument is selected to prepare the disc space and vertebral end plates for insertion of the fusion implant. The cutting depth of drilling instrument may be adjusted as desired. The drilling instrument is advanced into the intervertebral space adjacent to the first lateral side to shear the soft tissue and cut the bone of the adjacent vertebrae thereby forming a first bore “b1” which extends into the adjacent vertebrae “v1,v2” adjacent the first lateral side as depicted in FIG. 9. With the first bore drilled in the first lateral side, attention is directed to forming the bore in the second lateral side. With continued reference to FIG. 9, the second lateral side is accessed and the center entry point for the drill is identified. Preferably, the drill is positioned such that the second bore will overlap the first bore. The drill is activated to form the second bore “b2” of the intervertebral space. The first and second bores “b1,b2” may be tapped with a conventional tap instrument if desired.

With reference to FIG. 10, a first implant 100 is packed with bone growth inducing substances as is conventional in the art. The fusion implant 100 may then be mounted on an insertion instrument (not shown) and advanced within the intervertebral space by driving the implant 100 within the bore. Preferably, prior to insertion, the implant 100 is arranged such concave surfaces 128 extend in general parallel relation to the axis “s” of the spine, i.e., with the first group 120a of annular serrated portions adjacent the upper vertebrae “v1” and the second group of annular serrated portions adjacent the lower vertebrae “v2”. Upon insertion, the first and second groups 120,120b of serrations 120 engage the respective vertebrae “v1,v2” to secure implant body 102 within the intervertebral space “i”. Thereafter, the second implant 100 is positioned in the second bore “b2” in the same manner. Thus, the concave surface arrangement permits two implants 100, 100 to be placed in side-by-side arrangement. As appreciated, the concave surface arrangement reduces the effective cross-sectional dimension of implant 100 thereby facilitating placement of the implants in a restricted vertebral location.

Alternatively, as depicted in FIG. 11, the second implant may be replaced with a conventional threaded cylindrical implant “m” such as the implant disclosed in the Ray '373 patent. (FIG. 2) As appreciated, although the second bore overlaps the first bore, the clearance provided by the concave surface arrangement of the first implant 100 permits the second implant “m” to be rotated and advanced within the intervertebral space without interference. The second implant 100 is arranged such that the outer convex surface is received within the concave surface area of the first implant in nested side-by-side relation as shown.

Implants 100 form struts across the intervertebral space “i” to maintain the adjacent vertebrae “V₁ V₂” in appropriate spaced relation during the fusion process. Over a period of time, the adjacent vertebral tissue communicates through the apertures within implants 100 to form a solid fusion. Desirably, lateral vertebral tissue growth into the implant 100 is restricted due to the concave surface areas of the implant being devoid of apertures. Such lateral growth would inhibit the fusion process and potentially restrict subsequent spinal mobility.

While the above description contains many specifics, these specifics should not be construed as limitations on the scope of the disclosure, but merely as exemplifications of preferred embodiments thereof. For example, the fusion implant 100 could also be used for thoracic and cervical vertebrae. Those skilled in the art will envision many other possible variations that are within the scope and spirit of the disclosure as defined by the claims appended hereto.

What is claimed is:

1. An apparatus for facilitating fusion of adjacent bone structures, which comprises:
an implant body dimensioned for positioning between adjacent bone structures to maintain the bone structures in desired spaced relation during interbody fusion, the implant body defining a longitudinal axis and having an outer wall extending along the longitudinal axis, the outer wall having a plurality of annular serrated portions spaced along the longitudinal axis, the annular serrated portions dimensioned and configured to engage the adjacent bone structures to facilitate retention of the implant member therewithin, the implant member including at least one concave wall surface at least partially extending along the longitudinal axis, wherein the transverse cross-sectional dimension along a first transverse axis inclusive of the concave surface is less than the transverse cross-sectional dimension along a second transverse axis.

2. The apparatus according to claim 1 wherein the one concave wall surface extends substantially along the length of the implant member.

3. The apparatus according to claim 1 including a pair of diametrically opposed concave wall surfaces.

4. The apparatus according to claim 1 including a first group of annular serrated portions, and a second group of annular serrated portions in diametrical opposed relation to the first group, each of the serrated portions of the first and second groups subtending an angle less than 180 relative to the central longitudinal axis and being interconnected by the one concave wall surface.

5. The apparatus according to claim 1 wherein each serrated portion defines an entry surface and a trailing surface, the, trailing surface being obliquely arranged with respect to the longitudinal axis and being dimensioned to engage the adjacent bone structures in substantially locking relation therewith.

6. The apparatus according to claim 1 wherein the implant body includes an internal cavity defined within the outer wall for accommodating bone growth inducing substances.

7. The apparatus according to claim 6 wherein the implant body includes a plurality of apertures extending through the outer wall in communication with the internal cavity.

8. The apparatus according to claim 7 wherein the apertures extend through valleys defined between adjacent serrated portions in communication with the inner cavity to permit immediate contact of the vertebral bone tissue and the bone growth inducing substances within the inner cavity upon insertion of the implant body.

9. The apparatus according to claim 1 wherein the implant body defines entry and trailing longitudinal end faces, at least one of the end faces having an aperture therein in communication with the internal cavity.

10. The apparatus according to claim 9 further including an end cap, the end cap being mountable to the one end face to, enclose the internal cavity.

11. The apparatus according to claim 10 wherein each of the leading and trailing end faces include an aperture extending to communicate with the internal cavity.

12. The apparatus according to claim 1 wherein the implant body is generally cylindrical.

13. The apparatus according to claim 1 wherein the implant body is dimensioned and configured for positioning between adjacent vertebrae and to support the vertebrae in adjacent spaced relation.

14. A method for fusion of adjacent vertebrae, comprising the steps of:
accessing the intervertebral space defined between adjacent vertebrae;
forming at least partially overlapping first and second bores within the adjacent vertebrae and spanning the intervertebral space;
positioning a first implant within the first bore within the adjacent vertebrae, the first implant including an implant body defining a longitudinal axis and an outer wall extending along the longitudinal axis, the outer wall having a plurality of spaced annular serrated portions and a concave surface at least partially extending along the longitudinal axis, the implant body being arranged within the intervertebral space whereby the serrated portions engage the adjacent vertebrae in locking relation therewith and the concave surface is adjacent the second bore; and
positioning a second implant within the second bore within the adjacent vertebrae.

15. The method according to claim 14 wherein the second implant includes an implant body defining a longitudinal axis and an outer wall extending along the longitudinal axis, the outer wall having a plurality of spaced annular serrated portions and a concave surface at least partially extending along the longitudinal axis, and wherein the step of positioning the second implant includes arranging the implant body within the intervertebral space whereby the serrated portions engage the adjacent vertebrae in locking relation therewith and the concave surface is adjacent the first bore.

16. The method according to claim 15 wherein the steps of positioning include driving the implant bodies of the first and second implants within the respective first and second bores within the adjacent vertebrae.

17. The method according to claim 14 wherein the second implant includes a cylindrical implant body having an arcuate outer wall portion, and wherein the step of positioning the second implant includes arranging the implant body of the second implant whereby the arcuate outer wall portion of the second implant is correspondingly received within the concave surface of the first implant in nested relation therewith.

18. The method according to claim 17 wherein the implant body of the second implant includes an external threaded configuration and wherein the step of positioning the second implant includes rotating the implant body whereby the threaded configuration engages the adjacent vertebrae and is advanced within the second bore.

19. The method according to claim 14 wherein the implant bodies of the first and second implants each define an internal cavity for reception of bone growth inducing substances and have apertures extending through the exterior walls in communication with the internal cavity, and further including the step of permitting bone growth through the apertures to facilitate fusion of the adjacent vertebrae.
